(19) United States
(12) Patent Application Publication
(10) Pub. No.: US 2019/0076307 A1
(43) Pub. Date: Mar. 14, 2019

(54) ABSORBENT ARTICLE

(71) Applicant: UNICHARM CORPORATION, Shikokuchuo-shi, Ehime (JP)

(72) Inventors: Reiko TAKASHIMA, Kanonji-shi, Kagawa (JP); Hiroki MORI, Kanonji-shi, Kagawa (JP); Sakiko WATANABE, Kanonji-shi, Kagawa (JP)

(21) Appl. No.: 16/084,562

(22) PCT Filed: Feb. 22, 2017
(86) PCT No.: PCT/JP2017/006609

§ 371 Date: Sep. 13, 2018

(30) Foreign Application Priority Data
Mar. 17, 2016 (JP) 2016-053574

(51) Int. Cl. A61F 13/535; A61F 13/494; A61F 13/539; A61F 13/514

(52) U.S. Cl. CPC A61F 13/535; A61F 13/494; A61F 13/496; A61F 13/51456; A61F 13/51474; A61F 13/539

(57) ABSTRACT

A disposable diaper has an absorber including. The absorber includes a first region including a center of the absorber, a pair of second regions between the first folding lines and the second folding lines inward of the first folding lines, and a pair of third regions outward of the second folding lines and on the skin contact surface side of the second regions. Outer side edges of the third regions are outward of the first folding lines. The absorbent article includes a first bonded portion in which the first and second regions are bonded, a third bonded portion in which the third regions are bonded with the non-skin surface sheet, and a third non-bonded portion inward of the third bonded portion and outward of the first folding lines. The third non-bonded portion are across the center of the absorbent article in the crotch region.

### TECHNICAL FIELD

The present invention relates to an absorbent article including an absorber folded along folding lines extending in the front-back direction.

Disposable diapers including absorbers folded along folding lines extending in the front-back direction have been provided. In Patent Literature 1, an absorber is folded inward in a width direction along first lines extending in the front-back direction, and the folded portions are then folded outward in the width direction along second folding lines (e.g., see FIGS. 12 to 14 of Patent Literature 1).

### CITATION LIST

### Patent Literature

Patent Literature 1: JP 2005-517461 T

### SUMMARY OF INVENTION

When such an absorbent article is put on a wearer, the absorbent article is sandwiched between legs of the wearer and receives inward force in the width direction from the outside of the absorber. Applying the inward force in the width direction to the absorber has sometimes disturbed in maintaining the folding structure along the first and second folding lines. When the folding structure of the absorber is not maintained, the absorber cannot smoothly take in excretions and leakage of excretions may occur. In addition, applying the inward force in the width direction to the absorber may break or deform the absorber in an unintended manner, so that the absorber may not be disposed at a proper position relative to the wearer and leakage of excretions may occur.

The present disclosure is made in view of the above problem, and it is an object of the present disclosure to decrease occurrence of leakage of excretions in an absorbent article including an absorber that is folded along folding lines extending in the front-back direction.

An absorbent article (disposable diaper 10) of the present disclosure has a front-back direction (front-back direction L) and a width direction (width direction W) orthogonal to the front-back direction, and includes a front waistline region (front waistline region 20), a rear waistline region (rear waist line region 30), a crotch region (crotch region 25) located between the front waistline region and the rear waistline region, an absorber (absorber 40) extending in the front-back direction and including a pair of first folding lines (first folding lines FL1) and a pair of second folding lines (second folding lines FL2) for folding the absorber, and a non-skin surface sheet (backsheet 60a) located on a non-skin contact surface side of the absorber, in which the absorber includes, in a folded state in which the absorber is folded, a first region (first region 401) located between the pair of the first folding lines and including a center of the absorber in a width direction, a pair of second regions (second regions 402) located between the first folding lines and the second folding lines located inward of the first folding lines in the width direction, and disposed on a skin contact surface side of the first region, and a pair of third regions (third regions 403) located outward of the second folding lines in the width direction and disposed on the skin contact surface side of the second regions, outer side edges of the third regions being located outward of the first folding lines in the width direction, and the absorbent article includes, in the folded state in which the absorber is folded, a first bonded portion (first bonded portion R1) in which the skin contact surface of the first region is bonded with the non-skin contact surface of the second regions, a third bonded portion (third bonded portion R3) in which the third regions are bonded with the non-skin surface sheet, and a third non-bonded portion (third non-bonded portion NR3) located inward of the third bonded portion in the width direction and outward of the first folding lines in the width direction in which the third regions are not bonded with the non-skin surface sheet, the third non-bonded portion being provided across the center of the absorbent article in the front-back direction (center 1LC of the disposable diaper 10 in the front-back direction), and extending in the front-back direction in the crotch region.

### BRIEF DESCRIPTION OF DRAWINGS

FIG. 1 is a plan view of a disposable diaper according to an embodiment.

FIG. 2 is a cross-sectional view of the disposable diaper illustrated in FIG. 1 cut along line A-A.

FIG. 3 is a cross-sectional view of the disposable diaper illustrated in FIG. 1 cut along line B-B.

FIG. 4 is a plan view of a disposable diaper according to an embodiment.

FIG. 5 is an exploded plan view of an absorber.

FIG. 6 is a cross-sectional view of the absorber cut along line C-C of FIG. 5.

FIG. 7 is a cross-sectional view schematically illustrating a worn state of the disposable diaper according to the embodiment.

FIG. 8 is a cross-sectional view schematically illustrating a worn state of the disposable diaper according to a modification.

### DESCRIPTION OF EMBODIMENTS

According to the present specification and the accompanying drawings, at least the following matters will be disclosed.

An absorbent article includes: a front-back direction; a width direction orthogonal to the front-back direction; a front waistline region;

a rear waistline region; a crotch region located between the front waistline region and the rear waistline region; an absorber including a pair of first folding lines and a pair of second folding lines extending in the front-back direction and the first folding lines, the first folding lines and the second folding lines being provided to fold the absorber; and a non-skin surface sheet located on a non-skin contact surface side of the absorber.

The absorber includes, in a folded state in which the absorber is folded, a first region located between the pair of the first folding lines and including a center of the absorber in a width direction, a pair of second regions located between the first folding lines and the second folding lines located inward of the first folding lines in the width direction, and disposed on a skin contact surface side of the first region, and

a pair of third regions located outward of the second folding lines and disposed on the skin contact surface side of the second regions, outer side edges of the third regions being located outward of the first folding lines in the width direction. The absorbent article includes, in the folded state, a first bonded portion in which the skin contact surface of the first region is bonded with the non-skin contact surface of the second regions, a third bonded portion in which the third regions are bonded with the non-skin surface sheet, and a third non-bonded portion located inward of the third bonded portion in the width direction and outward of the first folding lines in the width direction and in which the third regions are not bonded with the non-skin surface sheet, the third non-bonded portion being provided across the center of the absorbent article in the front-back direction, and extending in the front-back direction in the crotch region.

An absorbent article is sandwiched between the legs of a wearer when put on the wearer. The absorbent article receives inward force from the outside in the width direction. Since the outer side edges of the third regions are located outside the first folding lines in the width direction, the inward force in the width direction is applied from the outside in the width direction to the outer side edges of the third regions. The third regions are deformed to absorb the inward force in the width direction from the outside in the width direction. This prevents deformation of the first region by such force. In addition, the folding structure along the first folding lines can be maintained easily due to bonding between the first and second regions. This prevents unintended deformation of the first region and the width reduction of the entire absorber, allowing the absorber to be disposed with a fixed width in the crotch region of the wearer. The absorber having the fixed width can absorb excretions and decrease leakage of excretion.

Meanwhile, the third regions and the non-skin surface sheet are bonded at the third bonded portion, but not bonded at the third non-bonded portion. When the inward force in the width direction is applied from the outside in the width direction, a region located inside the third bonded portion in the width direction is deformed to rise toward a wearer side from the third bonded portion as the base point. Thus, the second non-bonded portion, the third non-bonded portion, and the third bonded portion can form space surrounded by the third regions, the non-skin surface sheet, and the first region at least in the crotch region. The space can store urine. In addition, the space can diffuse urine in the front-back direction and keeps the body fluid in the entire area of the absorber in the front-back direction. The absorber can absorb the body fluid in the entire area and decrease leakage of the body fluid even when the absorber is used for long time. Further, the space can diffuse moisture in the front-back direction, decreasing stuffiness and improving wearing feeling.

The absorbent article preferably includes a second non-bonded portion in which the skin contact surface of the second regions is not boded with the non-skin contact surface of the third regions in a worn state in which the absorbent article is put on a wearer.

Since the skin contact surface of the second regions is not boded with the non-skin contact surface of the third regions in the worn state in the absorbent article, the third regions located on the right and left sides of the second regions bonded with the first region are deformed to rise toward the wearer side from the second regions as base points when the inward force in the width direction is applied from the outside in the width direction, thus forming the space on the non-skin contact surface side of the third regions. The space can hold excretions and decreases leakage of the excretions.

The absorbent article includes an elastic member stretchable in the front-back direction and a pair of uprising gathers disposed on both sides in a width direction of the center of the disposable diaper in the width direction. The third bonded portion is preferably disposed in a region overlapping the inner side edge of the elastic member.

In the absorbent article, the inner side edge of the uprising gather stretches in accordance with stretching of the elastic member, and rises toward the wearer side. The absorber located in a region overlapping the inner side edge of the elastic member is hardly deformed because the absorber is bonded with the non-skin surface sheet at the third bonded portion. The shape of the absorber can be maintained easily during contraction of the uprising gathers, so that the uprising gathers can easily rise toward the wearer side from the absorber. A large space for holding excretions can be formed between the uprising gathers and the absorber to decrease leakage of the excretions. The leakage of the excretions can be effectively decreased, while the folding structure of the absorber is maintained.

The absorbent article preferably includes a fourth bonded portion in which the third region is bonded with the non-skin surface sheet on both sides of the third non-bonded portion in the front-back direction.

Since the third regions are bonded with the non-skin surface sheet on both sides of the third non-bonded portion in the front-back direction in the absorbent article, the space between the third regions and the non-skin surface sheet minimizes leakage of the body fluid to the outside of the absorber in the front-back direction when the body fluid is diffused over the entire area of the absorber in the front-back direction. Thus, the body fluid is absorbed in the entire area of the absorber in the front-back direction and the leakage of the excretions can be decreased.

In the absorbent article, the length in the front-back direction of the fourth bonded portion located foreside of the third non-bonded portion is longer than a length in the front-back direction of the fourth bonded portion located rear side of the third non-bonded portion.

Urine is usually discharged in front of the center of the absorbent article in the front-back direction. Thus, urine tends to be held in the third non-bonded portion located in front of the center of the absorbent article in the front-back direction. Since the fourth bonded portion located in front of the center of the absorbent article in the front-back direction has a relatively long length in the front-back direction, the body fluid can be held in the third non-bonded portion located in the front portion, while leakage of urine held in the third non-bonded portion from the front edge of the absorber can be decreased.

In the absorbent article, a distance between the rear edge of the third non-bonded portion and the center of the absorbent article in the front-back direction is preferably longer than a distance between the front edge of the third non-bonded portion and the center of the absorbent article in the front-back direction.

Since the length of the third non-bonded portion behind the center of the absorbent article in the front-back direction is relatively long in the absorbent article, urine discharged in the front portion can be diffused backward through the third non-bonded portion. Thus, the excretions can be absorbed in the entire area of the absorber in the front-back direction, allowing the absorbing capacity to be used effectively and decreasing the leakage.

In the absorbent article, the first region does not need to be bonded with the non-skin surface sheet in the region inward from the outer side edges of the first region in the width direction.

When the inward force in the width direction is applied from the outside in the width direction to deform the right and left third regions to rise toward the wearer side in the absorbent article, the outer side edges of the first region can also be deformed easily to rise toward the wearer side. This allows a larger space surrounded by the third region, the non-skin surface sheet, and the second region to be formed.

In the absorbent article, the pair of the second folding lines is preferably apart from each other in the width direction.

In the absorbent article, only the first region is disposed between the pair of second folding lines of the absorber, and the first, second, and third regions are located outside the pair of second folding lines in the width direction. Thus, the absorber is relatively thin between the pair of second folding lines of the absorber, and has a recess toward the non-skin contact surface side compared to the regions outside the pair of second folding lines in the width direction. Since the absorber has the recess toward the non-skin contact surface side in the center in the width direction, space extending in the front-back direction can be formed between the wearer and the absorber. Such space enables diffusion of the body fluid in the front-back direction to more easily use the absorbing performance effectively over the entire region of the absorber in the front-back direction.

When the third region is deformed to rise toward the wearer side by the inward force in the width direction applied from the outside in the width direction, relatively high walls can be formed by the third regions on both sides across the space between the pair of second folding lines. Thus, the lateral leakage of the body fluid can be decreased, and the leakage of the excretions can further be decreased.

### Absorbent Article of Embodiment

An embodiment of a disposable diaper as an absorbent article according to the present invention will be described below by referring to the accompanying drawings. In the drawings, the same or similar parts are indicated by the same or similar reference symbols. The drawings are illustrated schematically, and dimensional ratios and other variables differ from those of actual measurements. The specific measurements or the like, therefore, should be determined by referring to the following description. The drawings may include different relationships or ratios of measurements.

### (1) Overview of Entire Structure of Disposable Diaper

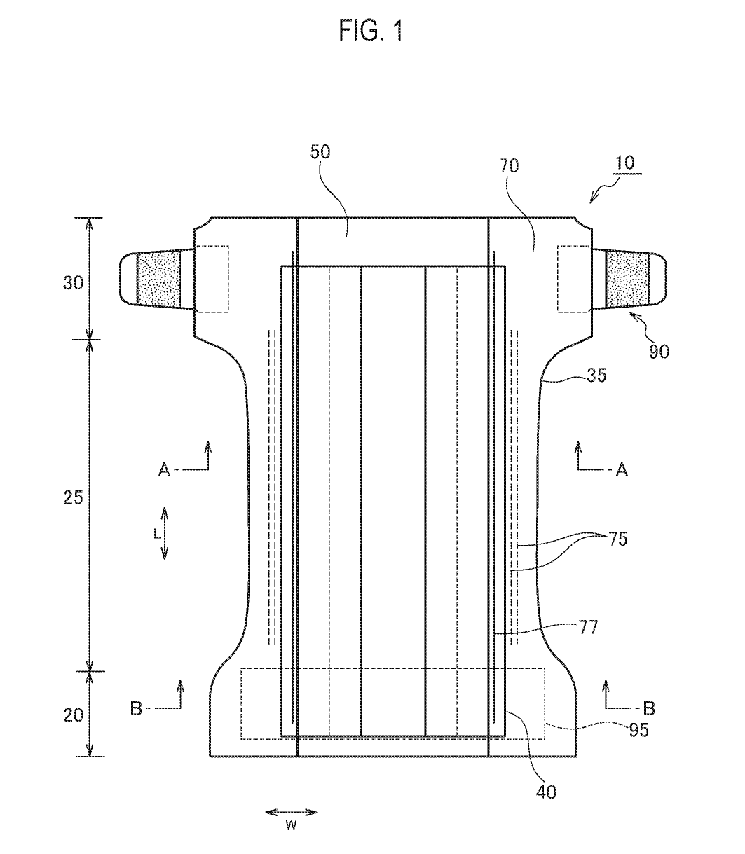

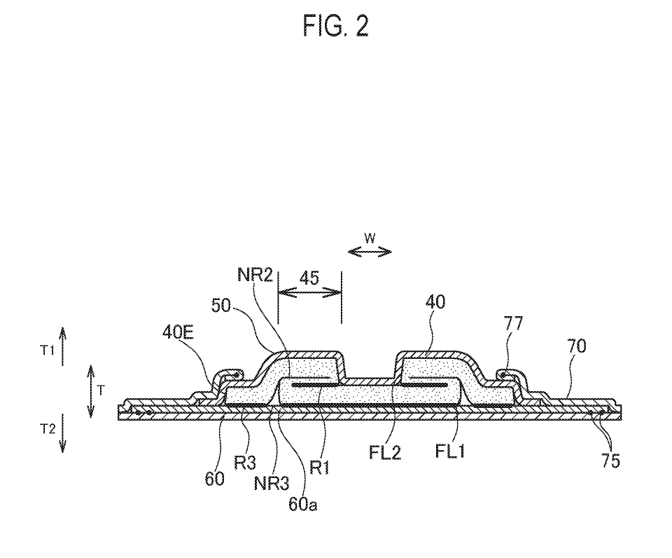

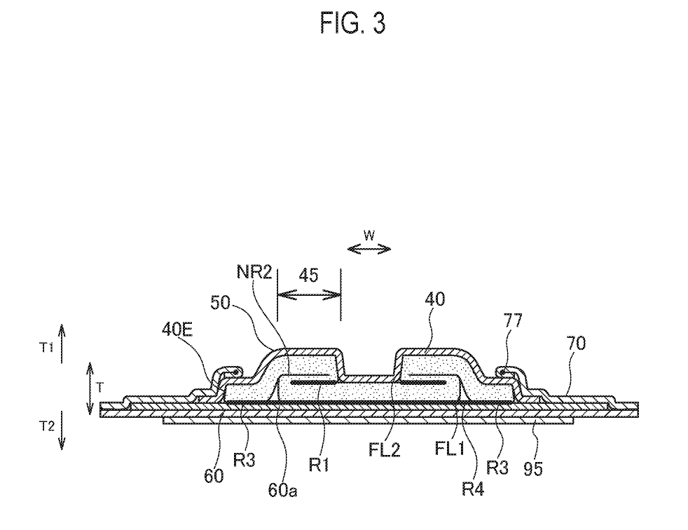

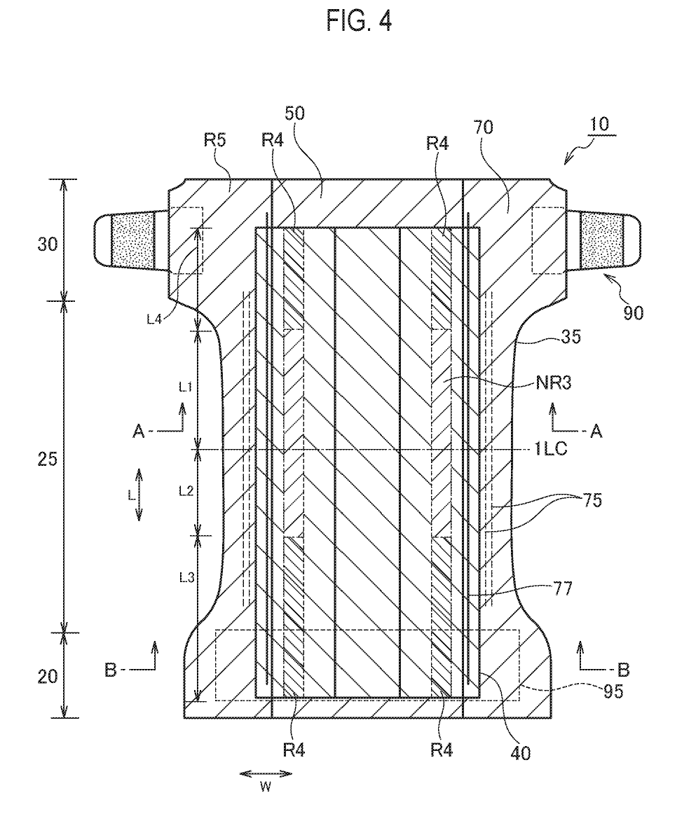

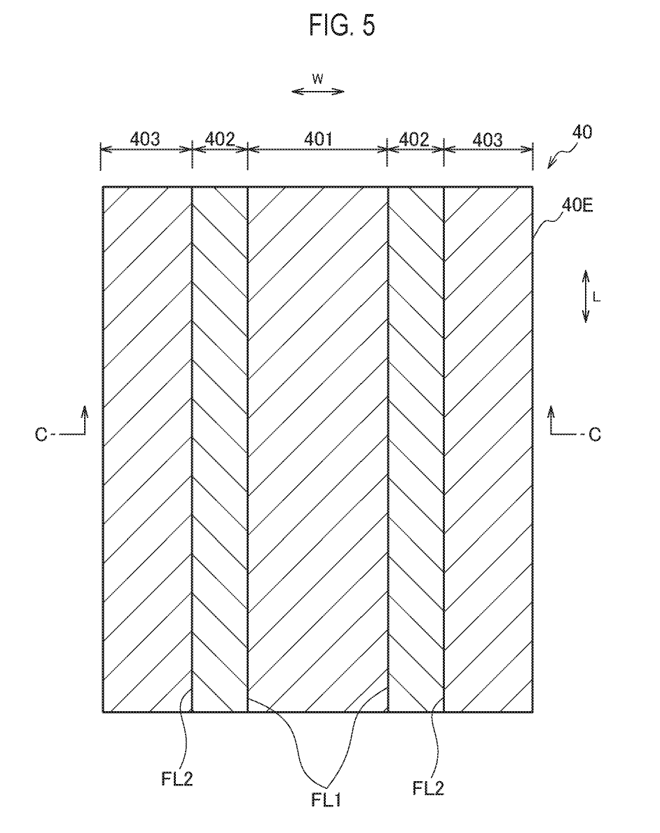

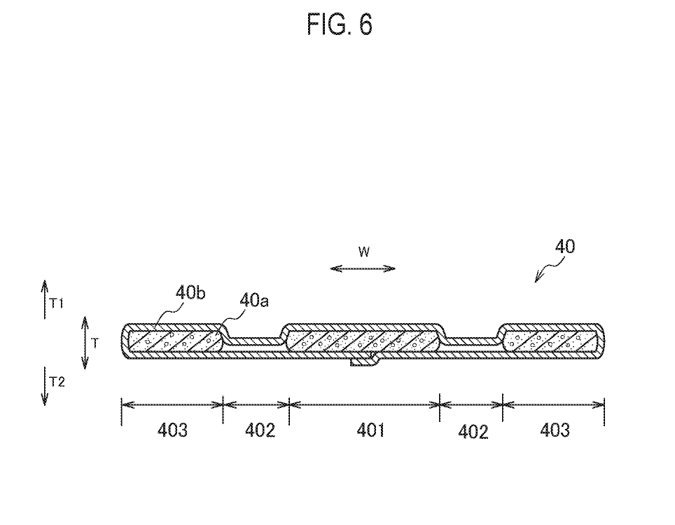

FIG. 1 is an exploded plan view of a disposable diaper 10 according to the present embodiment. FIG. 2 is a cross-sectional view of the disposable diaper 10 illustrated in FIG. 1 cut along line A-A. FIG. 3 is a cross-sectional view of the disposable diaper illustrated in FIG. 1 cut along line B-B. FIG. 4 is an exploded plan view of the disposable diaper illustrated in FIG. 1. FIG. 5 is an exploded plan view of an absorber. FIG. 6 is a cross-sectional view of the absorber illustrated in FIG. 5 cut along line C-C. FIGS. 1 and 4 are plan views illustrating leg expandable portions 75 and elastic members 77 of uprising gathers stretched until no wrinkle is formed in constituent components such as a topsheet 50 and side sheets 70 of a disposable diaper.

The disposable diaper 10 includes a front waistline region 20, a crotch region 25, and a rear waistline region 30. The front waistline region 20 is a region that comes in contact with a front waistline region (ventral part) of a wearer. The rear waistline region 30 is a region that comes in contact with a rear waistline region (dorsal part) of a wearer. The crotch region 25 is a region located between the front waistline region 20 and the rear waistline region 30 and includes leg-surrounding opening portions 35. The leg-surrounding opening portions 35 are formed on the outer side edges of the disposable diaper located around the leg-surrounding portions of the wearer when the disposable diaper is put on the wearer. The outer side edges represent outer ends in the width direction and the inner side edges represent inner ends in the width direction.

In the present embodiment, a front-back direction L is a direction from the front waistline region 20 toward the rear waistline region 30, a width direction W is a direction orthogonal to the front-back direction L, and a thickness direction T covers a skin contact surface side T1 relative to a wearer and a non-skin contact surface side T2 relative to a wearer opposite to the skin contact surface side T1.

The disposable diaper 10 at least includes an absorber 40, a skin surface sheet disposed on the skin contact surface side T1 of the absorber 40, and a non-skin surface sheet disposed on the non-skin contact surface side T2 of the absorber 40. The absorber 40 is disposed at least in the crotch region 25. As illustrated in FIG. 6, the absorber 40 includes an absorbent core 40a and a core wrap 40b. The absorbent core 40a can be made of a material similar to the material used in disposable diapers of the past including an absorbent material such as ground pulp or super-absorbent polymer (SAP), and made appropriately with a known member or material.

The core wrap 40b is a sheet covering the absorbent core 40a which is an absorbent material. The core wrap 40b is made of various types of fiber nonwoven cloths or tissues having a liquid-permeable characteristic. The absorber 40 will be described in detail later.

The skin surface sheet includes a topsheet 50 and side sheets 70. The topsheet 50 is formed of a liquid-permeable sheet, such as nonwoven fabric or woven fabric. The topsheet 50 is a sheet that comes into contact with the skin of the wearer.

The side sheets 70 cover the outer side edges of the topsheet 50 and extend farther outward in the width direction beyond the topsheet 50. An inner side edge of each side sheet 70 is folded outward in the width direction, and an elastic member 77 that is stretchable in the front-back direction is disposed between the two layers of the side sheet 70. The side sheet 70 and the elastic member 77 form uprising gather. The uprising gather includes the elastic member 77 that is stretchable in the front-back direction, and a pair of uprising gathers is disposed on both sides in the width direction of the center of the disposable diaper in the width direction.

The non-skin surface sheet at least includes a backsheet 60a and an exterior sheet 60. The backsheet 60a is liquid-impermeable and is disposed on the non-skin contact surface side T2 of the absorbent core 40a and the core wrap 40b. The exterior sheet 60 is disposed on the non-skin contact surface side T2 of the backsheet 60a.

The side sheets 70 each include a fastening tape 90. Each fastening tape 90 extends in the width direction W in the rear waistline region 30 and is fastened to a target portion 95 of the front waistline region 20 to hold the disposable diaper 10 on the body of the wearer. A pair of fastening tapes 90 is configured to be attached to the target portion 95. The absorber 40 includes the leg expandable portions 75 formed around the leg-surrounding opening portions 35 and stretchable in the front-back direction L on the outer sides of the absorber 40 in the width direction.

### (2) Structure of Absorber

Next, the structure of the absorber 40 is described in detail. As illustrated in FIGS. 5 and 6, the absorber 40 is a sheet-like material in the exploded state. The absorber 40 includes a pair of first folding lines FL1 extending in the front-back direction, and a pair of second folding lines FL2 extending in the front-back direction. The pair of first folding lines FL1 and the pair of second folding lines FL2 are provided to fold the absorber, and formed in a folded state of the absorber. The absorber 40 is folded four times along the pair of first folding lines FL1 and the pair of second folding lines FL2 to form multiple layers in a laminated manner. The pair of first folding lines FL1, the pair of second folding lines FL2, and the first folding lines FL1 and the second folding lines FL2 is apart from each other in the width direction. As illustrated in FIG. 5, the second folding lines FL2 are located outside the first folding lines FL1 in the width direction when the absorber 40 is exploded (or not folded along the first and second folding lines FL1, FL2). In a folded state of the absorber (folded along the first and second folding lines FL1, FL2), as illustrated in FIGS. 2 and 3, the second folding lines FL2 are located inside the first folding lines FL1 in the width direction.

In the folded state, the absorber 40 includes a first region 401 located between the pair of first folding lines FL1, a pair of second regions 402 each located between the first and second folding lines FL1, FL2, and a pair of third regions 403 each located outside the second folding line FL2 in the width direction. In the exploded state illustrated in FIGS. 5 and 6, the first region 401 is a region that includes the center of the absorber 40 in the width direction W and is located between the pair of first folding lines FL1. The second regions 402 are located on both sides of the first region 401 and between the first and second folding lines FL1 and FL2 in the exploded state. The third regions 403 are located on both sides of the first region 401 and the pair of second regions 402 to be regions outside the second folding lines FL2 in the width direction in the exploded state. The first, second, and third regions 401, 402, and 403 are continuously provided in the width direction W. The first, second, and third regions 401, 402, and 403 are covered by a continuous core wrap 40b. The core wrap 40b covers the first, second, and third regions 401, 402, 403.

The absorber 40 is valley-folded relative to the wearer along the first folding lines FL1, and then mountain-folded relative to the wearer along the second folding lines FL2. When the absorber 40 is folded, the second regions 402 are located on the skin contact surface side T1 of the first region 401, and the third regions 403 are located on the skin contact surface side T1 of the second regions 402. At least part of the third regions 403 need to be located on the skin contact surface side T1 of the second regions 402. The absorber 40 is folded along the first and second folding lines FL1, FL2 to achieve compact dimensions in the width direction.

The pair of second folding lines FL2 is apart from each other in the width direction W in the folded state. Overlapping portions 45 each including the first, second, and third regions 401, 402, and 403 are formed between the first and second folding lines FL1, FL2. The overlapping portion 45 is a portion in which the first, second, and third regions 401, 402, and 403 are layered, and located between the first and second folding lines FL1, FL2. Only the first region 401 is disposed between the pair of second folding lines FL2. A thickness of the absorber 40 is relatively thin between the pair of second folding lines FL2, and a recess is formed toward the non-skin contact surface side T2 compared to other regions outside the pair of second folding lines FL2 in the width direction. The recess in the center of the absorber 40 in the width direction W toward the non-skin contact surface side T2 allows space to be formed extending in the front-back direction between the wearer and the absorber. Such space enables diffusion of the body fluid in the front-back direction to more easily use the absorbing performance effectively over the entire region of the absorber in the front-back direction.

The third regions 403 are deformed to rise toward the wearer side when the inward force in the width direction W is applied from the outside in the width direction W, relatively high walls can be formed by the third regions 403 on both sides of the space between the pair of second folding lines FL2. Thus, the lateral leakage of the body fluid can be decreased, and the leakage of the excretions can further be decreased.

Outer side edges of the third regions 403 are located outside the first folding lines FL1 in the width direction W. Thus, only the third regions 403 are disposed outside the first folding lines FL1 in the width direction. In the folded state, outer side edges 40E of the absorber 40 are disposed at leg-surrounding portions of the wearer. The outer side edges 40E of the absorber 40 are the outer side edges of the third regions 403. In the folded state, only the third regions 403 are disposed at the outer side edges 40E of the absorber 40, so that the outer side edges 40E are relatively thin. This can reduce uncomfortable feeling of the wearer at the leg-surrounding portions.

The absorbent material of the second regions 402 has a basis weight lower than a basis weight of the absorbent material of the first region 401 and also lower than a basis weight of the absorbent material in the third regions 403. Specifically, the first region 401 includes pulp and SAP, while the second regions 402 do not substantially include any absorbent material. A design basis weight of the absorbent material such as the SAP is zero in the second regions 402, but the SAP or pulp may be mixed in from, for example, the first region in the manufacturing process. Thus, the basis weight of the pulp in the first region 401 and the basis weight of the pulp in the third regions 403 are higher than the basis weight of the pulp in the second regions 402. The second regions 402 may include pulp having a lower basis weight than the pulp used in, for example, the first region 401, or may only include the core wrap 40b instead of the pulp and SAP to achieve a relatively low basis weight. This allows smooth introduction of the body fluid to the first region 401 via the second regions 402. In a modification, the absorbent material in the second regions 402 has the same basis weight as the absorbent material in the first region 401 or the third regions 403.

Since the basis weight of the pulp in the second regions 402 is lower than the basis weight of the pulp in the first region 401 and the third regions 403, the overlapping portions 45 can be decreased from becoming excessively thick, and the body fluid can be introduced easily to the first region 401 located on the non-skin contact surface side T2. In addition, the basis weight of the pulp in the first regions 401 is higher than the basis weight of the pulp in the second region 402, so that the movement of the body fluid from the second regions 402 to the first region 401 can be facilitated, and the body fluid can be introduced easily to the first region 401 located on the non-skin contact surface side T2. A difference in rigidity is generated between the first and second folding lines FL1, FL2 according to the difference in the basis weight of the pulp. This facilitates folding of the absorber 40 along the first and second folding lines FL1, FL2 as base points.

The thickness of the second regions 402 is smaller than the thickness of the first region 401 and the thickness of the third regions 403. The first folding lines FL1 and the second folding lines FL2 are borderlines at which the thickness and basis weight of the absorber 40 change and at which the rigidity changes. Thus, the absorber 40 can be folded easily along the first and second folding lines FL1, FL2 as base points.

The topsheet 50 is disposed over the skin contact surface side T1 of the first region 401 located between the pair of second folding lines FL2 and the skin contact surface side T1 of the third regions 403. Since the topsheet 50 is disposed over the first region 401 and the third regions 403, the overlapping portions 45 hardly move inward in the width direction when the inward force in the width direction is applied to the overlapping portions 45, thus effectively decreasing deformation of the absorber 40. Specifically, the topsheet that extends in the width direction from the skin contact surface side T1 of the first region 401 extends toward the skin contact surface side T1 of the third regions 403 without extending between the first and second regions 401, 402. The topsheet 50 is not disposed between the first and second regions 401, 402 nor between the second and third regions 402, 403 in the overlapping portions 45. The folding structure of the absorber can be maintained. The excretions can be absorbed efficiently in the three regions including the first, second, and third regions 401, 402, 403 to decrease leakage of the excretions.

### (3) Bonding Manner of Absorber and Bonding Manner of Absorber and Non-Skin Surface Sheet

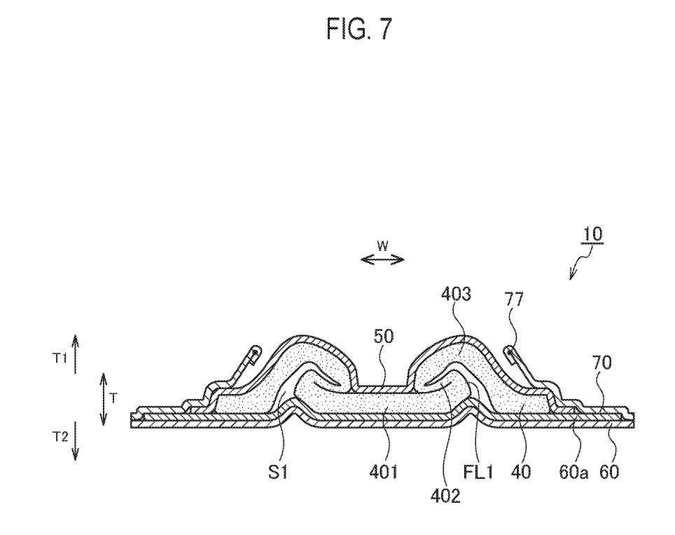

Next, a bonding manner of the absorber and a bonding manner of the absorber and the non-skin surface sheet are described. In the folded state in which the absorber 40 is folded, the disposable diaper 10 includes a first bonded portion R1, a second non-bonded portion NR2, a third bonded portion R3, a third non-bonded portion NR3, and a fourth bonded portion R4. In the worn state in which the absorbent article is worn, the disposable diaper 10 includes the second non-bonded portion NR2 (See FIG. 7). The bonded portions are regions in which an adhesive, such as a hot-melt adhesive, is disposed. The non-bonded portions are regions in which the adhesive such as a hot-melt adhesive is not disposed or the adhesive having an adhesive strength smaller than the adhesive strength of the bonded portions is disposed. The fact that the first bonded portions R1, the second non-bonded portions NR2, the third bonded portions R3, the third non-bonded portions NR3, and the fourth bonded portion R4 are provided can decrease unintended deformation of the disposable diaper 10 in the worn state, and decrease leakage of excretions. FIG. 7 is a cross-sectional view schematically illustrating the worn state of the disposable diaper. The disposable diaper 10 is sandwiched between the legs of the wearer when worn on the wearer. The disposable diaper 10 receives inward force in the width direction from the outside in the width direction. Since the outer side edges of the third regions 403 are located outside the first folding lines in the width direction, the inward force in the width direction from the outside in the width direction is applied on the outer side edges of the third regions 403. The third regions 403 are deformed to absorb the inward force in the width direction from the outside in the width direction. This prevents deformation of the first region 401 caused by such force.

The first bonded portions R1 are portions in which the skin contact surface of the first region 401 is bonded with the non-skin contact surface of the second regions 402. FIGS. 2 and 3 illustrate the range of the first bonded portions R1 in the width direction. The first bonded portions R1 may be provided on the entire surface or part of the surface between the skin contact surface of the first region 401 and the non-skin contact surface of the second regions 402. The first bonded portions R1 are provided to decrease displacement of the position of the first region 401 and the second regions 402, allowing the folding structure along the first folding lines FL1 to be maintained easily when the inward force in the width direction is applied from the outside in the width direction to the disposable diaper 10. This prevents unintended deformation of the first region 401 and width reduction of the entire absorber, allowing the absorber 40 to be disposed within a certain width range relative to the crotch region of the wearer. The absorber 40 having a certain width range can absorb the excretions and decrease leakage of the excretions.

The second non-bonded portions NR2 are portions in which the skin-contact surface of the second regions 402 is not bonded with the non-skin contact surface of the third regions 403. The second non-bonded portions NR2 are provided over the entire surface between the skin contact surface of the second regions 402 and the non-skin contact surface of the third regions 403 at least in the crotch region 25. The second non-bonded portions NR2 are provided across a center 1LC of the disposable diaper 10 in the front-back direction to extend in the front-back direction in the crotch region 25. The second non-bonded portions NR2 of the present embodiment are regions in which no adhesive is disposed and remain in the non-bonded state in the folded state. A concept of the second non-bonded portions NR2, however, includes the structure in which the skin contact surface of the second regions 402 is bonded with the non-skin contact surface of the third region 403 in the folded state, and the second non-bonded portions NR2 become non-bonded earlier than the first bonded portions when the inward force in the width direction from the outside in the width direction is applied in the worn state. Since the skin contact surface of the second regions 402 is not bonded with the non-skin contact surface of the third regions 403 in the crotch region 25 in the worn state, the right and left third regions 403 are deformed to rise toward the wearer side along the second regions 402 bonded with the first region 401 as base points when the inward force in the width direction from the outside in the width direction is applied, thus forming space on the non-skin contact surface side T2 of the third regions 403. The space between the second and third regions 402, 403 can hold the excretions and decrease leakage of the excretions.

The disposable diaper 10 may include second bonded portions in which the skin contact surface of the second regions 402 is bonded with the non-skin contact surface of the third regions 403 in the rear waistline region 30 and the front waistline region 20. The second bonded portions are located outside the second non-bonded portions in the front-back direction. The space to store the excretions can be provided in the crotch region 25 even when the second bonded portions are provided in the rear waistline region 30 and the front waistline region 20, thus achieving the effect of preventing leakage of the excretions.

The third bonded portions R3 are portions in which the third regions 403 are bonded with the non-skin surface sheet. Specifically, the third bonded portions R3 include the non-skin contact surface of the third regions 403 bonded with the skin contact surface of the backsheet 60a which is the non-skin surface sheet. The third regions 403 are provided in a range covering the entire region of the absorber 40 in the front-back direction L. The third bonded portions R3 are provided within a certain range from the outer side edges of the third regions 403 to the inside in the width direction W. The third bonded portions R3 are located outside of and apart from the first folding lines FL1 in the width direction W. The third non-bonded portion NR3 is provided inside the third bonded portions R3 in the width direction W. The third non-bonded portions NR3 are portions in which the third regions 403 are not bonded with the non-skin surface sheet. The third regions 403 are disposed in the crotch region 25 and not in the front waistline region 20 and the rear waistline region 30. The third non-bonded portions NR3 are provided across the center 1LC of the disposable diaper 10 in the front-back direction and extend in the front-back direction L in the crotch region 25. The third non-bonded portions NR3 are provided inside the third bonded portions R3 in the width direction W and outside the first folding lines FL1 in the width direction W. The first region 401 is bonded with the backsheet 60a in a region inside the first folding lines FL1 in the width direction.

The third regions 403 and the backsheet 60a are bonded at the third bonded portions R3, but not bonded at the third non-bonded portions NR3. When the inward force in the width direction W is applied from the outside in the width direction W, the absorber 40 that is in contact with the third non-bonded portions NR3 is deformed to rise toward the wearer side from the third bonded portions R3 as base points. This allows space S1 surrounded by the third regions 403, the backsheet 60a, and the first region 401 to be formed at least in the crotch region 25 (see FIG. 7). The space S1 can store urine. The space S1 can also diffuse urine in the front-back direction L to hold the body fluid in the entire region of the absorber 40 in the front-back direction L. Even when the absorber 40 is used for long time, the entire region of the absorber 40 can absorb the body fluid and decrease leakage of the body fluid. In addition, the space S1 can diffuse moisture in the front-back direction L and decrease stuffiness, thus improving the wearing feeling.

The third bonded portions R3 are disposed in a region overlapping the inner side edges of the elastic members 77. When the uprising gather includes a plurality of elastic members, the inner side edge of the elastic members 77 represents the inner side edge of the elastic member located innermost side in the width direction. The inner side edge of the elastic members 77 of the uprising gathers contracts in accordance with stretching and contraction of the elastic members 77 and rises toward the wearer side. A portion of the absorber overlapping the inner side edge of the elastic members 77 is bonded with the backsheet 60a at the third bonded portion R3 and is hardly deformed. This facilitates the shape of the absorber 40 to be kept during contraction of the uprising gathers, and allows the uprising gathers to rise easily from the absorber 40 toward the wearer side. Thus, a wide space can be formed between the uprising gathers and the absorber 40 to store the excretions and decrease leakage of the excretions. As a result, the leakage of the excretions can be decreased effectively, while the folding structure of the absorber 40 is maintained.

A distance L1 between the rear edge of the third non-bonded portions NR3 and the center 1LC of the disposable diaper 10 in the front-back direction L is longer than a distance L2 between the front edges of the third non-bonded portions NR3 and the center 1LC of the disposable diaper 10 in the front-back direction. Since the length of the third non-bonded portions NR3 is relatively long behind the center of the disposable diaper 10 in the front-back direction L, it is possible to diffuse urine that is discharged in the front side backward through the third non-bonded portions NR3. Thus, the absorber 40 can absorb the excretions in the entire region of the absorber 40 in the front-back direction L and effectively use the absorbing capacity to decrease leakage.

Fourth bonded portions R4 are portions in which the third regions 403 are bonded with the backsheet 60a. Specifically, the non-skin contact surface of the third regions 403 is bonded with the backsheet 60a as the non-skin surface sheet in the fourth bonded portions R4. The fourth bonded portions R4 are located on both sides of the third non-bonded portions NR3 in the front-back direction. The fourth bonded portions R4 are located in the front-back direction in a range from the outer edges of the third non-bonded portions NR3 to the outer edges of the absorber 40. The fourth bonded portions R4 are disposed in the width direction in regions sandwiched between the inner side edges of the third bonded portions R3 and the first folding lines FL1. Since the third regions 403 are bonded with the backsheet 60a on both sides of the third non-bonded portions NR3 in the front-back direction, the space between the third regions 403 and the backsheet 60a decreases leakage of the body fluid outside the absorber in the front-back direction even when the body fluid in the space is diffused over the entire region in the front-back direction. Thus, the body fluid is absorbed in the entire area of the absorber in the front-back direction and the leakage of the excretions can be decreased.

The fourth bonded portions R4 located foreside of the third non-bonded portions NR3 have a length L3 in the front-back direction which is longer than a length L4 of the fourth bonded portions R4 in the front-back direction located rear side of the third non-bonded portions NR3. At least part of the fourth bonded portions R4 located in front of the third non-bonded portions NR3 is provided in the front waistline region 20. At least part of the fourth bonded portions R4 located behind the third non-bonded portions NR3 is provided in the rear waistline region 30. Usually, urine is discharged in front of the center 1LC of the disposable diaper 10 in the front-back direction. Thus, urine tends to be held in the third non-bonded portion located in front of the center of the absorbent article in the front-back direction. Since the fourth bonded portions located in front of the center of the disposable diaper 10 in the front-back direction is relatively long in the front-back direction, the body fluid can be held in the third non-bonded portions located in front, while leakage of urine in the third non-bonded portions from the front edge of the absorber can be decreased.

Fifth bonded portions R5 formed by bonding the skin surface sheet with the non-skin surface sheet are provided outside the absorber both in the width direction and the front-back direction. The fifth bonded portions are provided in a region between the outer side edges of the absorber in the front-back direction and the outer side edges of the disposable diaper 10 in the front-back direction, and a region between the outer side edges of the absorber and the outer side edges of the disposable diaper 10. The fifth bonded portions R5 prevent leakage of the absorbent material (especially SAP) of the absorber and the body fluid from the front, rear, and outer side edges of the disposable diaper 10.

### (4) Absorbent Article of Modification

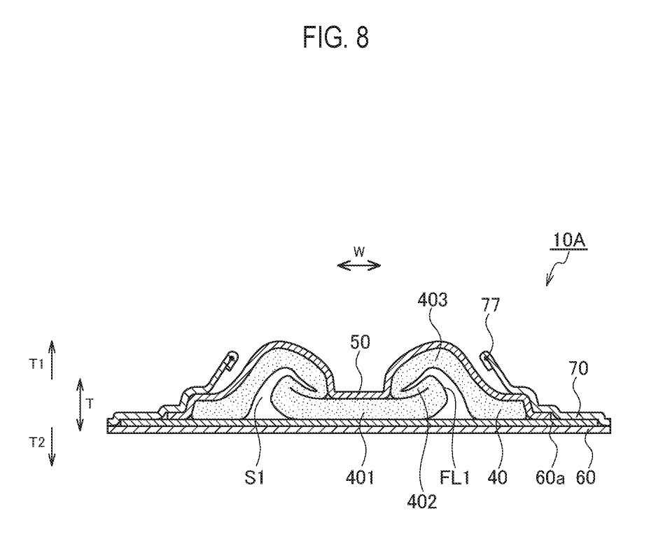

Next, a disposable diaper 10A as an absorbent article according to a modification is described by referring to FIG. 8. In the description of the modification, the constituent components similar to those in the above-described embodiment are indicated by the same reference symbols and the description thereof will not be repeated. FIG. 8 is a cross-sectional view of a disposable diaper 10A in the width direction according to a first modification. In the disposable diaper 10A according to the first modification, a first region 401 of an absorber 40 is not bonded with a backsheet 60a which is the non-skin surface sheet in a region inside the outer side edges of the first region 401 in the width direction W. The first region 401 is bonded with the non-skin surface sheet in the center region of the first region 401 in the width direction.

When the inward force in the width direction is applied from the outside in the width direction W and the right and left third regions 403 are deformed to rise toward the wearer side, the disposable diaper 10A according to the first modification facilitates deformation of the outer side edges of the first region 401 to rise toward the wearer side. This allows a large space S1 surrounded by the third regions 403, the non-skin surface sheet, and the second region 402 to be formed. The space S1 stores excretions and decreases leakage of the excretions by diffusing the excretions in the front-back direction.

Although the embodiment of the present invention has been described above to disclose the subject matter of the present invention, the description and the accompanying drawings that constitute a part of the disclosure are in no way intended to limit the scope of the invention. Apparently, persons who have ordinary skill in the art would devise various other embodiments, examples, and operational techniques from this disclosure. In view of the above, therefore, the technical range of the present invention shall be defined appropriately by only the specified matters defined in the claims of the present invention.

The entire contents of Japanese Patent application No. 2016-053574 (filed on Mar. 17, 2016) have been incorporated into the description by reference.

### INDUSTRIAL APPLICABILITY

Provided is the absorbent article including the absorber folded along the folding lines extending in the front-back direction and capable of decreasing occurrence of leakage.

### REFERENCE SIGNS LIST

10, 10A: Absorbent article
20: Front waistline region
25: Crotch region
30: Rear waistline region
40: Absorber
40a: Absorbent core
40b: Core wrap
401: First region
402: Second region
403: Third region
50: Topsheet
60: Exterior sheet
60a: Backsheet
L: Front-back direction
T: Thickness direction
W: Width direction
FL1: First folding line
FL2: Second folding line
R1: First bonded portion
NR2: Second non-bonded portion
R3: Third bonded portion
NR3: Third non-bonded portion
R4: Fourth bonded portion
R5: Fifth bonded portion

1. An absorbent article comprising:
a front-back direction;
a width direction orthogonal to the front-back direction;
a front waistline region;
a rear waistline region;
a crotch region located between the front waistline region and the rear waistline region;
an absorber including a pair of first folding lines and a pair of second folding lines extending in the front-back direction and the first folding lines, the first folding lines and the second folding lines being provided to fold the absorber; and
a non-skin surface sheet located on a non-skin contact surface side of the absorber, wherein
the absorber includes, in a folded state in which the absorber is folded,
a first region located between the pair of the first folding lines and including a center of the absorber in a width direction,
a pair of second regions located between the first folding lines and the second folding lines located inward of the first folding lines in the width direction, and disposed on a skin contact surface side of the first region, and
a pair of third regions located outward of the second folding lines and disposed on the skin contact surface side of the second regions,
outer side edges of the third regions being located outward of the first folding lines in the width direction, and
the absorbent article includes, in the folded state,
a first bonded portion in which the skin contact surface of the first region is bonded with the non-skin contact surface of the second regions,
a third bonded portion in which the third regions are bonded with the non-skin surface sheet, and
a third non-bonded portion located inward of the third bonded portion in the width direction and outward of the first folding lines in the width direction and in which the third regions are not bonded with the non-skin surface sheet, the third non-bonded portion being provided across the center of the absorbent article in the front-back direction, and extending in the front-back direction in the crotch region.

2. The absorbent article according to claim 1, further comprising:
a second non-bonded portion in which the skin contact surface of the second region is not bonded with the non-skin contact surface of the third regions in a worn state in which the absorbent article is worn.

3. The absorbent article according to claim 1, further comprising:
a pair of upraising gathers each having an elastic member stretching in the front-back direction and disposed on both sides in the width direction of a center of the absorbent article in the width direction, wherein
the third bonded portion is disposed in a region overlapping an inner side edge of the elastic member.

4. The absorbent article according to claim 1, further comprising:
a fourth bonded portion in which the third region is bonded with the non-skin surface sheet, the fourth bonded portion being disposed on both sides of the third non-bonded portion in the front-back direction.

5. The absorbent article according to claim 4, wherein
the length in the front-back direction of the fourth bonded portion located foreside of the third non-bonded portion is longer than a length in the front-back direction of the fourth bonded portion located rear side of the third non-bonded portion.

6. The absorbent article according to claim 1, wherein
a distance between a rear edge of the third non-bonded portion and the center of the absorbent article in the front-back direction is longer than a distance between a front edge of the third non-bonded portion and the center of the absorbent article in the front-back direction.

7. The absorbent article according to claim 1, wherein
the first region is not bonded with the non-skin surface sheet in a region inward of the outer side edges of the first region in the width direction.

8. The absorbent article according to claim 1, wherein
the pair of second folding lines is apart from each other in the width direction.
